(19) United States
(12) Patent Application Publication
(10) Pub. No.: US 2023/0270844 A1
(43) Pub. Date: Aug. 31, 2023

(54) MULTIVALENT BETA-CORONAVIRUS VACCINES, THEIR DESIGNS AND USES

(71) Applicant: Greffex, Inc., Houston, TX (US)

(72) Inventors: Uwe D. STAERZ, Denver, CO (US); Janae Wheeler CULL, Brighton, CO (US); Daniel F. PRESTON, Denver, CO (US); Yan QI, Lone Tree, CO (US)

(21) Appl. No.: 18/016,344

(22) PCT Filed: Jul. 22, 2021
(86) PCT No.: PCT/US2021/042795

§ 371 Date: Jan. 15, 2023

Related U.S. Application Data
(60) Provisional application No. 63/055,139, filed on Jul. 22, 2020.

(51) Int. Cl. A61K 39/215

(52) U.S. Cl. CPC A61K 39/215; A61K 2039/5256

(57) ABSTRACT

A multivalent vaccine for preventing CoV infection includes more than one protein antigen derived from antigens encoded within a CoV genome. At least one of the more than one protein antigen derived from antigens encoded within a CoV genome is a protein antigen, RNA-encoded genetic information, DNA-encoded genetic information, or genetic information within a genetic vector.

### CROSS REFERENCE TO RELATED APPLICATIONS

This application claims priority to and is a non-provisional application of Provisional Application No. 63,055,139, filed on Jul. 22, 2020, which is incorporated herein by reference in its entirety.

### FIELD

The disclosure relates to β-coronavirus vaccines, and more particularly to the design and assembly of specific antigens to induce immune responses against β-coronavirus infections.

### BACKGROUND

Coronaviruses (CoVs) are classified into four genera: alpha-, beta-, gamma- and delta-coronaviruses. β-CoVs are enveloped, positive-strand RNA (30 kb) viruses capable of infecting mammals, generally bats and rodents, though many β-CoVs are known to infect humans as well. The virus enters the host cell via the angiotensin-converting enzyme 2 (ACE2). Of their four major structural proteins, the S protein mediates cell receptor binding. It is divided into the S1 and S2 chains which are separated by a furan cut site. The SARS receptor binding domain (RBD) is located in S1, and the membrane fusion section is located in S2. Other major proteins include the M, N and envelop (E) proteins.

Infections with CoVs in humans and animals commonly produce mild to moderate upper-respiratory tract illnesses of short duration. Exceptions are the Severe Acute Respirator Syndrome (SARS-1), the Middle East Respiratory Syndrome (MERS) and the Wuhan-originating SARS-CoV-2 (SARS-2) (also referred to as COVID-19) that are characterized by severe and often lethal symptoms. The first cases of MERS were reported in Saudi Arabia in September 2012, with major eruptions in 2014 and 2015, followed by small seasonal outbreaks. 2,494 cases of confirmed MERS have so far been observed resulting in deaths in 858 patients (34.3% lethality; WHO). The first cases of SARS-2 infections were seen in December 2019. As of Apr. 16, 2020, there were an estimated 632,000 cases reported and an estimated 31,000 deaths in the United States alone, as reported by the Center for Disease Control (CDC), resulting in a 4.9% lethality. SARS-2 is highly infectious to humans, with an R₀ estimated around 3 (Liu 2020). The World Health Organization (WHO) declared the SARS-2 worldwide pandemic a Global Health Emergency on Jan. 30, 2020.

In the United States, SARS-2 has been reported in all 50 states, Washington D.C., and at least 4 territories. Outbreaks in long-term care facilities and homeless shelters have emphasized the risk of exposure and infection in congregate settings. Person-to-person transmission either direct or through droplets is assumed to be the primary means of transmission of SARS-2.

While SARS-2 generally presents as a mild illness, with the most common symptoms being fever, cough or chest tightness, and dyspnoea, the disease is most fatal for older and polymorbid patients. Severe complications include pneumonia, hypercoagulation, multiorgan dysfunction (including myocardial injury and kidney) and ultimately death. In children, multisystem inflammatory syndrome (MIS-C) is a serious condition in which some body parts, such as the heart, blood vessels, kidneys, digestive system, brain, skin or eyes, become inflamed.

Specific treatments for SARS-2 are not available but under investigation. The present recommendation is to observe patients with asymptomatic or mild illness. The best approach to prevent further spread of the disease is the development of specific vaccines. Besides inactivated virus vaccines, different approaches of engineered vaccines are being investigated with an emphasis on RNA-based and virally vectored vaccines and genetic vaccines. Most of these vaccine use the SARS-2 spike (S) protein as the principal antigen. Data from animal testing as well as early clinical trials have been released. Prime/boost immunization protocols were required to induce strong neutralizing antibody responses. Many patients, however, would prefer a single-dose regimen to a multi-dose or booster-requiring regime. A need exists to investigate whether engineered vaccines with more than a single antigen further enhance immune protection after a single administration.

CoVs induce both humoral and cellular immune responses. Animal, as well as clinical, studies demonstrate that SARS-1 and MERS infections raise potent neutralizing antibody responses against the S protein. Further, SARS-2 humoral responses similarly targeted the S protein with other antibodies binding to the M protein. The M protein also serves as a focus of CD8⁺T cell responses. Anti-SARS-2 CD4⁺T cells principally see both the N and the S antigen. Inactivated virus vaccines are inherently multivalent. They may provide stronger SARS-2 responses than single S protein vaccines. Animal studies have suggested that inactivated virus vaccines are prone to the induction of Th2-type possibly anti-N disease enhancing immune responses. Disease enhancement was also observed with S-based component vaccines, yet they were not evident with virally vectored anti-S vaccines. The FDA prefers SARS-2 vaccines that demonstrate Th1-type T cell polarization together with strong neutralizing antibodies.

Overall mutation rates of SARS-related (SARSr) viruses have been calculated at 0.1 mutations/generation. Minor changes in the S receptor binding domain of animal SARSr viruses can enhance binding to the human ACE2 and therefore facilitate a jump to the human population. Aligning S protein sequences reveals significant divergence throughout the gene with significant stable areas within the S2 region, whereas the M and N of the SARSr viruses show a significantly lower mutation rate overall. Therefore, multivalent vaccines will provide better protection against SARS-2 variants.

Current vaccines engineered as Ad vectors have repeatedly demonstrated higher and mores sustained immunogenicity in comparison with other vaccine systems. Minimally modified early generation (eg) Ad vectors carry numerous endogenous Ad genes, against which vigorous humoral and cellular immune responses are induced. Therefore prime/boost vaccinations regimens with egad vaccines relied on vaccines of different design for the second dose. Yet, in a recent clinical trial an animal derived eg Ad vaccine saw increased immune responses after a boosted injection. Anti-Ad response have most efficiently been minimized by Ad vectors fully deleted (fd) of all endogenous Ad genes. Such fdAd vectors saw enhanced transgene expression, prolonged maintenance in vivo, and improved immunogenicity. The packaging information for fdAd genomes was originally delivered with second viral constructs, a hybrid baculovirus-adenovirus or a helper virus, which led to contaminations with replication competent Ad (RCA) or helper viruses.

To avoid these issues, helper virus-independent technology has been developed. Helper virus-independent vaccines minimize pre-existing and induced interfering anti-Ad responses by the full deletion of all endogenous genes and the packaging into capsids of rare stereotypes, such as human Ad6. Current helper virus-independent technology is built upon two independently-modifiable components—(i) the base vector modules able to accept transgene constructs of up to 33kb which carry the ITRs and a packaging signal, and (ii) different circular packaging plasmids based on the Ad2, Ad5, Ad6 and Ad35 stereotypes (pPaC2/5/6 and pPaB35). The base vector modules have all Ad genes removed and replaced by size-compensating stuffers derived from fragments of the human housekeeping gene 5-aminoimidazole-4-carboxamide ribonucleotide formyltrans-ferase gene (ATIC). In the circular packaging plasmids, the left ITR, the packaging signal, and the E1, E3 and protein IX genes are deleted. The vector modules are encapsulated by an optimized one-week co-transfection protocol using HEK-293-derived HTP7/Q7 packaging cells. It is desirable to use this same technology to provide CoV vaccines with potent immunogenicity and that require a single dose to provide protection against a wide range of CoVs.

### SUMMARY

In an embodiment, the disclosure provides a multivalent vaccine. In accordance with embodiments of the present disclosure, the multivalent vaccine for preventing CoV infection comprises more than one protein antigen derived from antigens encoded within a CoV genome.

In another embodiment, at least one of the more than one protein antigen derived from antigens encoded within a CoV genome are selected from the group consisting of a protein antigen, RNA-encoded genetic information, DNA-encoded genetic information, genetic information within a genetic vector, and combinations thereof In a further embodiment, at least one of the more than one protein antigens is a protein expressed on or by a CoV particle. In still a further embodiment, at least one of the more than one protein antigens is a protein expressed on or by a cell infected with CoV.

In yet a further embodiment, at least one of the more than one protein antigens is a protein obtained from a production cell transfected with CoV genetic information to produce the protein. In an embodiment, the production cell is a eukaryotic cell. In another embodiment, the production cell is a bacterium. In another embodiment, the production cell is a fungus.

In an embodiment, at least one of the more than one protein antigens is RNA-encoded genetic information which codes for the expression of the at least one of the more than one protein antigens. In another embodiment, at least one of the more than one protein antigens is DNA-encoded genetic information which codes for the expression of the at least one of the more than one protein antigens.

In an embodiment, at least one of the more than one protein antigens is genetic information within a genetic vector. In another embodiment, the genetic vector is a viral genetic vector. In still another embodiment, the viral genetic vector is selected from the group consisting of adenovirus associated virus vectors, adenoviral vectors, vaccinia vectors, polyoma virus vectors, alpha-virus vectors and combinations thereof. In a further embodiment, the viral genetic vector is a bacterium. In yet another embodiment, the genetic vector is a bacterial genetic vector.

In an embodiment, the CoV is an β-CoV. In a further embodiment, the β-CoV is selected from the group consisting of SARSr viruses, MERS viruses and combinations thereof. In still a further embodiment, the β-CoV is a SARSr virus. In another embodiment, the SARSr is selected from the group consisting of a SARS-1 virus, a SARS-2 virus, and combinations thereof. In a further embodiment, the SARSr virus is a SARS-2 virus. In still a further embodiment, the β-CoV is a MERS virus.

In an embodiment, the multivalent vaccine comprises at least three different protein antigens derived from antigens encoded within a CoV genome.

In an embodiment, at least one of the more than one protein antigens is derived from a protein selected from the group consisting of a CoV spike (S) protein, a CoV membrane (M) protein, a CoV nucleocapsid (N) protein, a CoV envelope (E) protein, a replicase 1a/1b protein, and ORF 4, 9, 10 and 13 encoded proteins. In an embodiment, at least one of the more than one protein antigens is derived from a CoV S protein. In another embodiment, at least one of the more than one protein antigens is derived from a CoV M protein. In a further embodiment, at least one of the more than one protein antigens is derived form a CoV N protein.

In an embodiment, the vaccine comprises at least one protein antigen derived from a CoV S protein and a least one protein antigen derived from a protein selected form the group consisting of a CoV M protein, a CoV N protein and a CoV E protein.

In an embodiment, the disclosure provides a method of stimulating an immune response in a subject. In accordance with embodiments of the present disclosure, the method of stimulating an immune response in a subject comprises administering to the subject an effective amount of a composition comprising more than one protein antigen derived from antigens encoded within a CoV genome.

In an embodiment, at least one of the more than one protein antigen derived from antigens encoded within a CoV genome are selected from the group consisting of a protein antigen, RNA-encoded genetic information, DNA-encoded genetic information, genetic information within a genetic vector, and combinations thereof. In another embodiment, at least one of the more than one protein antigens is a protein expressed on or by a CoV particle. In yet another embodiment, at least one of the more than one protein antigens is a protein expressed on or by a cell infected with CoV.

In an embodiment, at least one of the more than one protein antigens is a protein obtained from a production cell transfected with CoV genetic information to produce the protein. In a further embodiment, the production cell is a eukaryotic cell. In another embodiment, the production cell is a bacterium. In still a further embodiment, the production cell is a fungus.

In an embodiment, at least one of the more than one protein antigens is RNA-encoded genetic information which codes for the expression of the at least one of the more than one protein antigens. In an embodiment, at least one of the more than one protein antigens is DNA-encoded genetic information which codes for the expression of the at least one of the more than one protein antigens.

In an embodiment, at least one of the more than one protein antigens is genetic information within a genetic vector. In an embodiment, the genetic vector is a viral genetic vector. In another embodiment, the viral genetic vector is selected from the group consisting of adenovirus associated virus vectors, adenoviral vectors, vaccinia vectors, polyma virus vectors, alpha-virus vectors, and combinations thereof. In still another embodiment, the viral genetic vector is a bacterium. In a further embodiment, the genetic vector is a bacterial genetic vector.

In an embodiment, the CoV is an β-CoV. In a further embodiment, the β-CoV is selected from the group consisting of SARSr viruses, MERS viruses and combinations thereof. In yet a further embodiment, the β-CoV is a SARSr virus. In still another embodiment, the SARSr is selected from the group consisting of a SARS-1 virus, a SARS-2 virus, and combinations thereof. In a further embodiment, the SARSr virus is a SARS-2 virus. In still a further embodiment, the β-CoV is a MERS virus.

In an embodiment, the composition comprises at least three different protein antigens derived from antigens encoded within a CoV genome.

In an embodiment, at least one of the more than one protein antigens is derived from a protein selected from the group consisting of a CoV spike (S) protein, a CoV membrane (M) protein, a CoV nucleocapsid (N) protein, a CoV envelope (E) protein, a replicase 1a/1b protein, and ORF 4, 9, 10 and 13 encoded proteins. In another embodiment, at least one of the more than one protein antigens is derived from a CoV S protein. In yet another embodiment, at least one of the more than one protein antigens is derived from a CoV M protein. In a further embodiment, at least one of the more than one protein antigens is derived form a CoV N protein. In an embodiment, the composition comprises at least one protein antigen derived from a CoV S protein and a least one protein antigen derived from a protein selected form the group consisting of a CoV M protein, a CoV N protein and a CoV E protein.

In an embodiment, the subject is a mammal subject. In another embodiment, the subject is a human subject.

In an embodiment, the administering is by intramuscular, intradermal or subdermal injection. In a further embodiment, the administering is by oral or intranasal administration.

In an embodiment, the disclosure provides a multivalent vaccine for preventing CoV infection. In accordance with embodiments of the present disclosure, the multivalent vaccine for preventing CoV infection comprises more than one of (i) a protein antigen derived from antigens encoded within a first CoV genome, (ii) RNA-encoded genetic information which codes for the expression of a protein antigen derived from antigens encoded within the first CoV genome, (iii) DNA-encoded genetic information which codes for the expression of a protein antigen derived from antigens encoded within the first CoV genome, (iv) a genetic information within a genetic vector which codes for the express of a protein antigen derived from antigens encoded within the first CoV genome.

In an embodiment, the first CoV genome is selected from the group consisting of a SARSr genome and a MERS genome. In an embodiment, the vaccine further comprises more than one of (i) a protein antigen derived from antigens encoded within a second CoV genome, (ii) RNA-encoded genetic information which codes for the expression of a protein antigen derived from antigens encoded within the second CoV genome, (iii) DNA-encoded genetic information which codes for the expression of a protein antigen derived from antigens encoded within the second CoV genome, (iv) a genetic information within a genetic vector which codes for the express of a protein antigen derived from antigens encoded within the second CoV genome.

None.

### BRIEF DESCRIPTION OF THE DRAWINGS

None.

### DETAILED DESCRIPTION

Before any embodiments of the present disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The use of “including essentially” and “consisting essentially of” and variations thereof herein is meant to compass the items listed thereafter, as well as equivalents and additional items provided such equivalents and additional items to not essentially change the properties, use or manufacture of the whole. The use of “consisting of” and variations thereof herein is meant to include the items listed thereafter and only those items.

With reference to the drawings, like numbers refer to like elements throughout. It will be understood that, although the terms first, second, etc., may be used herein to describe various elements, components, regions, and/or sections, these elements, components, regions and/or sections should not be limited by these terms. These terms are used only to distinguish one element, component, region and/or section from another element, component, region and/or section. Thus, a first element, component, region or section could be termed a second element, component, region or section without departing from the disclosure.

The numerical ranges in this disclosure are approximate, and thus may include values outside of the range unless otherwise indicated. Numerical ranges include all values from and including the lower and the upper values (unless specifically stated otherwise), in increments of one unit, provided that there is a separation of at least two units between any lower value and any higher value. As an example, if a compositional, physical or other property, such as, for example, amount of a component by weight, etc., is from 10 to 100, it is intended that all individual values, such as 10, 11, 12, etc., and sub ranges, such as 10 to 44, 55 to 70, 97 to 100, etc., are expressly enumerated. For ranges containing explicit values (e.g., a range from 1, or 2, or 3 to 5, or 6, or 7), any subrange between any two explicit values is included (e.g., the range 1-7 above includes subranges 1 to 2; 2 to 6; 5 to 7; 3 to 7; 5 to 6, etc.). For ranges containing values which are less than one or containing fractional numbers greater than one (e.g., 1.1, 1.5, etc.), one unit is considered to be 0.0001, 0.001, 0.01 or 0.1, as appropriate. For ranges containing single digit numbers less than ten (e.g., 1 to 5), one unit is typically considered to be 0.1. These are only examples of what is specifically intended, and all possible combinations of numerical values between the lowest value and the highest value enumerated, are to be considered to be expressly stated in this disclosure.

Spatial terms, such as “beneath,” “below,” “lower,” “above,” “upper,” and the like, may be used herein for ease of description to describe one element's or feature's relationship to another element(s) or feature(s) as illustrated in the figures. It will be understood that the spatially relative terms are intended to encompass different orientations depending on the orientation in use or illustration. For example, if a device in the figures is turned over, elements described as “below” or “beneath” other elements or features would then be oriented “above” the other elements or features. Thus, the exemplary term “below” can encompass both an orientation of above and below. A device may be otherwise oriented (rotated 90° or at other orientations) and the spatially relative descriptors used herein interpreted accordingly.

As used herein, the term “and/or” includes any and all combinations of one or more of the associated listed items. For example, when used in a phrase such as “A and/or B,” the phrase “and/or” is intended to include both A and B; A or B; A (alone); and B (alone). Likewise, the term “and/or” as used in a phrase such as “A, B and/or C” is intended to encompass each of the following embodiments” A, B and C; A, B, or C; A or C; A or B; B or C; A and C; A and B; B and C; A (alone); B (alone); and C (alone).

In an embodiment, the present disclosure provides a multivalent vaccine against CoV infections, the multivalent vaccine comprising more than one protein antigen derived from antigens encoded within a CoV genome.

In an embodiment, the more than one protein antigen derived from antigens encoded within a CoV genome is delivered as a protein in the vaccine, delivered as RNA-encoded genetic information in the vaccine, delivered as DNA-encoded genetic information in the vaccine, and/or delivered as genetic information within a genetic vector in the vaccine.

### Protein Antigens

In an embodiment, the vaccine comprises more than one CoV protein antigen derived from antigens encoded within a CoV genome. In an embodiment, the more than one protein antigen derived from antigens encoded within a CoV genome are (i) antigens expressed on or by the CoV (ii) antigens expressed on or by cells infected with the CoV, and/or (iii) antigens expressed on or by production cells engineered to produce the protein antigens.

When used herein, the term “more than one” is used to refer to at least two different components, e.g., two different protein antigens derived from antigens encoded within a CoV genome. In an embodiment “more than one” can be at least two, more than two, at least three, more than three, at least four, more than four, at least five, more than five, and so on. In a particular embodiment, the vaccine comprises from more than one, or at least two, or more than two, or at least three, or more than three, or at least four, or more than four, or at least five to more than five different protein antigens derived from antigens encoded within a CoV genome.

The CoV may be any CoV, including β-CoVs, SARSr viruses, and more particularly SARS-2 viruses. In an embodiment, the vaccine comprises more than one CoV protein antigen derived from antigens encoded within a CoV genome, wherein the more than one CoV protein antigens are each from the same CoV strain. In a particular embodiment, each CoV protein antigen is derived from antigens encoded within a CoV genome selected from the group consisting of a SARSr genome and a MERS genome, or more specifically a SARS-1 genome, a SARS-2 genome and a MERS genome. In another embodiment, the vaccine comprises more than one CoV protein antigen derived from a first CoV genome and more than one CoV protein antigen derived from a second CoV genome. For example, in an embodiment, the vaccine comprises more than one CoV protein antigen derived from a first CoV genome selected from the group consisting of a SARS-1 genome, a SARS-2 genome and a MERS genome and more than one CoV protein antigen derived from a second CoV genome selected from the group consisting of a SARS-1 genome, a SARS-2 genome and a MERS genome, wherein the first and second CoV genomes are not the same.

In another embodiment, the vaccine comprises more than one protein antigen derived from antigens encoded within a CoV genome, wherein a first protein antigen is derived from a first CoV genome and a second protein antigen is derived from a second CoV genome, wherein the first and second CoV genomes are different and each selected from the group consisting of a SARS-1 genome, a SARS-2 genome, and a MERS genome.

In an embodiment, the more than one protein antigen derived from antigens encoded within a CoV genome comprises at least one protein antigen derived from antigens encoded within a SARSr virus genome, or at least one protein antigen derived from antigens encoded within a SARS-2 virus genome. In another embodiment, the vaccine comprises two, or three, or more than three protein antigens derived from antigens encoded within a CoV genome, wherein one, some or all of the protein antigens are derived from antigens encoded within a SARSr virus genome, or a SARS-2 virus genome.

In an embodiment, the protein antigens are derived from a CoV membrane (M) protein, a nucleocapsid (N) protein, an envelope (E) protein, a replicase 1a/1b protein, and the ORF 4, 9, 10 and 13 encoded proteins.

### The Vaccine

At least one of the more than one protein antigen derived from antigens encoded within a CoV genome is delivered as a protein in the vaccine, delivered as RNA-encoded genetic information in the vaccine, delivered as DNA-encoded genetic information in the vaccine, and/or delivered as genetic information within a genetic vector in the vaccine.

In an embodiment, the protein antigen is delivered as a protein in the vaccine. The protein antigen may be a protein expressed on or by the CoV. In an embodiment, one, some or all of the more than one CoV protein antigen derived from antigens encoded within a CoV genome are expressed on or by the CoV.

In another embodiment, the protein antigen is expressed by a cell infected with the CoV. In an embodiment, one, some or all of the more than one CoV protein antigen derived from antigens encoded within a CoV genome are expressed by a cell infected with the CoV.

In an embodiment, the protein antigen is made by engineering production systems to produce the protein antigen in production cells using genetic information encoded within the CoV genome. Production cells can be bacteria or eukaryotic cells, including, but not limited to, animal cells and plant cells. Animal cells may be selected from human cells, insect cells, and cells of animals other than humans and insects. Plant cells include cells of living plants. In an embodiment, the production cells are selected from the group consisting of eukaryotic cells, bacteria cells, fungal cells, and combinations thereof. In an embodiment, one, some or all of the CoV protein antigens are made by engineering production systems to produce the protein antigen in production cells using genetic information encoded within the CoV genome.

To obtain the one or more CoV protein antigens expressed on or by the CoV for use in the vaccine, the protein antigens are extracted from purified CoV, or purified SARSr viruses, or purified SARS-2 viruses. In an embodiment, the protein antigens are used as a mixtures without further purification after extraction. In another embodiment, the extracted protein antigens are purified and used as a purified mixture. In still a further embodiment, the protein antigens may be purified and separated to form a custom mixture or used individually.

To obtain one or more CoV protein antigens expressed by a cell infected with the CoV, the protein antigens are extracted from CoV-infected cells, or SARSr-infected cells, or SARS-2-infected cells. In an embodiment, the CoV-infected cells are purified before the extraction. In an embodiment, the protein antigens are used as a mixture without further purification after extraction. In another embodiment, the extracted protein antigens are purified and used as a purified mixture. In still a further embodiment, the protein antigens may be purified and separated to form a custom mixture or used individually.

To obtain one or more CoV protein antigens made by engineering production systems to produce the protein antigen in production cells using genetic information encoded within the CoV genome, a production cell is transfected with genetic expression vectors that code for the at least one of the more than one CoV protein antigens. The protein antigens are then extracted from the production cells. In an embodiment, the protein antigens are used as a mixture without further purification after extraction. In another embodiment, the extracted protein antigens are purified and used as a purified mixture. In still a further embodiment, the protein antigens may be purified and separated to form a custom mixture or used individually.

In an embodiment, the vaccine comprises genetic information encoded within the CoV genome to engineer genetic constructs that code for the expression of the protein antigen. Such genetic constructs include, but are not limited to, RNA and DNA constructs. In an embodiment, one, some or all of the more than one protein antigen derived from antigens encoded within a CoV genome are delivered by the vaccine as RNA-encoded genetic information, DNA-encoded genetic information, and combinations thereof.

The creation of genetic constructs is known, and genetic constructs useful in the present vaccine may be obtained in similar means known in the art.

In embodiments in which the vaccine comprises genetic information encoded within the CoV genome to engineer genetic constructs that code for the expression of the protein antigen, the protein antigen is made by the vaccine recipient in response to receiving the vaccine with the genetic construct.

In an embodiment, the vaccine comprises genetic information encoded within the CoV genome to engineer expression vectors that carry transgene expression cassettes that code for the expression of the protein antigen. Such expression vectors are plasmid-type vectors and viral vectors, such as, but not limited to, adenoviral associated virus vectors, adenoviral vectors, SV40-derived vectors, VSV-type vectors, vaccinia-derived vector, and bacterial vectors. In a particular embodiment, the vaccine comprises the genetic information in a genetic vector. In a further embodiment, the genetic vector is selected from the group consisting of a viral genetic vector, a bacterial genetic vector, and combinations thereof. In an embodiment, the genetic vector is a viral vector selected from the group consisting of adenovirus associated virus vectors, adenoviral vectors, vaccinia vectors, polyoma virus vectors, alpha-virus vectors, and combinations thereof.

Exemplary vectors are described in PCT/US2021/28187 and PCT/US2021/31974, both incorporated in their entireties herein by reference.

In embodiments in which the vaccine comprises genetic information encoded within the CoV genome to engineer expression vectors that carry transgene expression cassettes that code for the expression of the protein antigen, the protein antigen is made by the vaccine recipient in response to receiving the vaccine with the expression vector.

As set forth previously, the vaccine comprises more than one protein antigen derived from antigens encoded within a CoV genome. In one embodiment, the at least one protein antigen is derived from a protein selected from the group consisting of a CoV spike (S) protein, a CoV membrane (M) protein, a CoV nucleocapsid (N) protein, a CoV envelope (E) protein, a replicase 1a/1b protein, and ORF 4, 9, 10 and 13 encoded proteins. In an particular embodiment, the vaccine comprises a protein antigen derived from a CoV S protein, a CoV M protein, and a CoV N protein. In a further embodiment, the vaccine comprises a protein antigen derived from a CoV S protein and at least one other protein antigen derived from a CoV M protein, a CoV N protein, and a CoV E protein.

### Method of Stimulating an Immune Response

In an embodiment, the disclose provides a method of stimulating an immune response in a subject. The method comprises administering to the subject an effective amount of a composition comprising more than one protein antigen derived from antigens encoded within a CoV genome. In an embodiment, the composition is a vaccine composition in accordance with any embodiment or combination of embodiments described herein.

The vaccine may be delivered to animal subjects, such as mammal subjects or, more specifically, human subjects, at defined doses and defined numbers of administrations as determined by the particular circumstances (i.e., “effective amounts”).

Vaccines may be administered by different routes, such as, but not limited to, intramuscular injection, subcutaneous injection, intracutaneous injections, oral administration and intranasal administration.

### Example

A bivalent CoV vaccine comprises a transgene expression cassette for the SARS-2 S antigen and a transgene expression cassette for the SARS-2 M antigen. It is anticipated that this vaccine will induce potent humoral (anti-S and anti-M) and cellular (CD4⁺ T cell: anti-S; CD8⁺T cell: anti-M) immune responses. The bivalent vaccine, as an adenovirally vectored vaccine, will demonstrate Th1-type T cell polarization without leading to an enhancement of the disease process upon a subsequent SARS-2 infection. The vector genome in the vaccine carries the transgene expression cassette that guides the expression of the human codon-optimized S and M antigens. The expression cassette is driven from a cytomegalovirus (CMV) immediate early promoter/enhancer and terminated by a poly-adenylation site derived from the human growth hormone (HGH) gene. The two transgenes are separated by a human encephalomyelitis virus internal ribosomal entry site (IRES).

It is contemplated that the include of a third antigen (the SARS-2 N antigen) will further enhance the efficacy of the vaccine compared to the bivalent vaccine.

While multiple embodiments of a vaccine have been described in detail herein, it should be apparent that modifications and variations thereto are possible, all of which fall within the true spirit and scope of the invention. In particular, while the vaccines have been described in detail with respect to β-CoVs, and more particularly SARSr viruses and the SARS-2 virus, it will be appreciated that the vaccines can be modified in accordance with the skill of one in the art to apply to other classes of coronaviruses, such as, for example, α-CoVs, γ-CoVs, and δ-CoVs. Further, since numerous modifications and changes will readily occur to those skilled in the art, it is not desired to limit the invention to the exact construction and operation shown and described, and accordingly, all suitable modifications and equivalents may be resorted to, falling within the scope of this disclosure.

What is claimed is:

1. A multivalent vaccine for preventing CoV infection comprising more than one protein antigen derived from antigens encoded within a CoV genome.

2. The multivalent vaccine of claim 1, wherein at least one of the more than one protein antigen derived from antigens encoded within a CoV genome are selected from the group consisting of a protein antigen, RNA-encoded genetic information, DNA-encoded genetic information, genetic information within a genetic vector, and combinations thereof.

3. The multivalent vaccine of claim 1, wherein at least one of the more than one protein antigens is a protein expressed on or by a CoV particle.

4. The multivalent vaccine of claim 1, wherein at least one of the more than one protein antigens is a protein expressed on or by a cell infected with CoV.

5. The multivalent vaccine of claim 1, wherein at least one of the more than one protein antigens is a protein obtained from a production cell transfected with CoV genetic information to produce the protein.

6. The multivalent vaccine of claim 5, wherein the production cell is a eukaryotic cell.

7. The multivalent vaccine of claim 5, wherein the production cell is a bacterium.

8. The multivalent vaccine of claim 5, wherein the production cell is a fungus.

9. The multivalent vaccine of claim 1, wherein at least one of the more than one protein antigens is RNA-encoded genetic information which codes for the expression of the at least one of the more than one protein antigens.

10. The multivalent vaccine of claim 1, wherein at least one of the more than one protein antigens is DNA-encoded genetic information which codes for the expression of the at least one of the more than one protein antigens.

11. The multivalent vaccine of claim 1, wherein at least one of the more than one protein antigens is genetic information within a genetic vector.

12. The multivalent vaccine of claim 11, wherein the genetic vector is a viral genetic vector.

13. The multivalent vaccine of claim 12, wherein the viral genetic vector is selected from the group consisting of adenovirus associated virus vectors, adenoviral vectors, vaccinia vectors, polyoma virus vectors, alpha-virus vectors and combinations thereof.

14. The multivalent vaccine of claim 12, wherein the viral genetic vector is a bacterium.

15. The multivalent vaccine of claim 11, wherein the genetic vector is a bacterial genetic vector.

16. The multivalent vaccine of claim 1, wherein the CoV is an β-CoV.

17. The multivalent vaccine of claim 16, wherein the β-CoV is selected from the group consisting of SARSr viruses, MERS viruses and combinations thereof.

18. The multivalent vaccine of claim 17, wherein the β-CoV is a SARSr virus.

19. The multivalent vaccine of claim 18, wherein the SARSr is selected from the group consisting of a SARS-1 virus, a SARS-2 virus, and combinations thereof.

20. The multivalent vaccine of claim 19, wherein the SARSr virus is a SARS-2 virus.

21. The multivalent vaccine of claim 17, wherein the β-CoV is a MERS virus.

22. The multivalent vaccine of claim 1, comprising at least three different protein antigens derived from antigens encoded within a CoV genome.

23. The multivalent vaccine of claim 1, wherein at least one of the more than one protein antigens is derived from a protein selected from the group consisting of a CoV spike (S) protein, a CoV membrane (M) protein, a CoV nucleocapsid (N) protein, a CoV envelope (E) protein, a replicase 1a/1b protein, and ORF 4, 9, 10 and 13 encoded proteins.

24. The multivalent vaccine of claim 23, wherein at least one of the more than one protein antigens is derived from a CoV S protein.

25. The multivalent vaccine of claim 23, wherein at least one of the more than one protein antigens is derived from a CoV M protein.

26. The multivalent vaccine of claim 23, wherein at least one of the more than one protein antigens is derived form a CoV N protein.

27. The multivalent vaccine of claim 1, wherein the vaccine comprises at least one protein antigen derived from a CoV S protein and a least one protein antigen derived from a protein selected form the group consisting of a CoV M protein, a CoV N protein and a CoV E protein.

28. A method of stimulating an immune response in a subject comprising administering to the subject an effective amount of a composition comprising more than one protein antigen derived from antigens encoded within a CoV genome.

29. The method of claim 28, wherein at least one of the more than one protein antigen derived from antigens encoded within a CoV genome are selected from the group consisting of a protein antigen, RNA-encoded genetic information, DNA-encoded genetic information, genetic information within a genetic vector, and combinations thereof.

30. The method of claim 28, wherein at least one of the more than one protein antigens is a protein expressed on or by a CoV particle.

31. The method of claim 28, wherein at least one of the more than one protein antigens is a protein expressed on or by a cell infected with CoV.

32. The method of claim 28, wherein at least one of the more than one protein antigens is a protein obtained from a production cell transfected with CoV genetic information to produce the protein.

33. The method of claim 32, wherein the production cell is a eukaryotic cell.

34. The method of claim 32, wherein the production cell is a bacterium.

35. The method of claim 32, wherein the production cell is a fungus.

36. The method of claim 28, wherein at least one of the more than one protein antigens is RNA-encoded genetic information which codes for the expression of the at least one of the more than one protein antigens.

37. The method of claim 28, wherein at least one of the more than one protein antigens is DNA-encoded genetic information which codes for the expression of the at least one of the more than one protein antigens.

38. The method of claim 28, wherein at least one of the more than one protein antigens is genetic information within a genetic vector.

39. The method of claim 38, wherein the genetic vector is a viral genetic vector.

40. The method of claim 39, wherein the viral genetic vector is selected from the group consisting of adenovirus associated virus vectors, adenoviral vectors, vaccinia vectors, polyma virus vectors, alpha-virus vectors, and combinations thereof.

41. The method of claim 39, wherein the viral genetic vector is a bacterium.

42. The method of claim 38, wherein the genetic vector is a bacterial genetic vector.

43. The method of claim 28, wherein the CoV is an β-CoV.

44. The method of claim 43, wherein the β-CoV is selected from the group consisting of SARSr viruses, MERS viruses and combinations thereof.

45. The method of claim 44, wherein the β-CoV is a SARSr virus.

46. The method of claim 45, wherein the SARSr is selected from the group consisting of a SARS-1 virus, a SARS-2 virus, and combinations thereof.

47. The method of claim 46, wherein the SARSr virus is a SARS-2 virus.

48. The method of claim 44, wherein the β-CoV is a MERS virus.

49. The method of claim 28, wherein the composition comprises at least three different protein antigens derived from antigens encoded within a CoV genome.

50. The method of claim 28, wherein at least one of the more than one protein antigens is derived from a protein selected from the group consisting of a CoV spike (S) protein, a CoV membrane (M) protein, a CoV nucleocapsid (N) protein, a CoV envelope (E) protein, a replicase 1a/1b protein, and ORF 4, 9, 10 and 13 encoded proteins.

51. The method of claim 50, wherein at least one of the more than one protein antigens is derived from a CoV S protein.

52. The method of claim 50, wherein at least one of the more than one protein antigens is derived from a CoV M protein.

53. The method of claim 50, wherein at least one of the more than one protein antigens is derived form a CoV N protein.

54. The method of claim 28, wherein the composition comprises at least one protein antigen derived from a CoV S protein and a least one protein antigen derived from a protein selected form the group consisting of a CoV M protein, a CoV N protein and a CoV E protein.

55. The method of claim 28, wherein the subject is a mammal subject.

56. The method of claim 55, wherein the subject is a human subject.

57. The method of claim 28, wherein the administering is by intramuscular, intradermal or subdermal injection.

58. The method of claim 28, wherein the administering is by oral or intranasal administration.

59. A multivalent vaccine for preventing CoV infection comprising more than one of (i) a protein antigen derived from antigens encoded within a first CoV genome, (ii) RNA-encoded genetic information which codes for the expression of a protein antigen derived from antigens encoded within the first CoV genome, (iii) DNA-encoded genetic information which codes for the expression of a protein antigen derived from antigens encoded within the first CoV genome, (iv) a genetic information within a genetic vector which codes for the express of a protein antigen derived from antigens encoded within the first CoV genome.

60. The multivalent vaccine of claim 59, wherein the first CoV genome is selected from the group consisting of a SARSr genome and a MERS genome.

61. The multivalent vaccine of claim 59, further comprising more than one of (i) a protein antigen derived from antigens encoded within a second CoV genome, (ii) RNA-encoded genetic information which codes for the expression of a protein antigen derived from antigens encoded within the second CoV genome, (iii) DNA-encoded genetic information which codes for the expression of a protein antigen derived from antigens encoded within the second CoV genome, (iv) a genetic information within a genetic vector which codes for the express of a protein antigen derived from antigens encoded within the second CoV genome.
